(19) United States
(12) Patent Application Publication
(10) Pub. No.: US 2026/0249283 A1
(43) Pub. Date: Aug. 27, 2026

(54) MOBILE PCR SYSTEMS, MOBILE REVERSE PCR HOODS AND RELATED METHODS

(71) Applicant: Radeas, LLC, Wake Forest, NC (US)

(72) Inventor: Phillip Radford, Wake Forest, NC (US)

(21) Appl. No.: 18/706,699

(22) PCT Filed: Nov. 1, 2022
(86) PCT No.: PCT/US2022/048593

§ 371 Date: Sep. 4, 2025

Related U.S. Application Data
(60) Provisional application No. 63/274,354, filed on Nov. 1, 2021.

(51) Int. Cl. B01L 1/00; A61L 9/20; B01D 46/00; B01D 46/62; B08B 13/00; B08B 15/02; B60P 3/00; B60S 1/64

(52) U.S. Cl. CPC B01L 1/52; A61L 9/20; B01D 46/0028; B01D 46/0032; B01D 46/0047; B01D 46/62; B08B 13/00; B08B 15/023; B60S 1/64; B01D 2273/30; B01D 2279/51; B01D 2279/65; B01L 2200/02; B01L 2200/185; B01L 2300/0681; B08B 2215/003; B60P 3/00

(57) ABSTRACT

Mobile polymerase chain reaction (PCR) hoods, mobile PCR systems, and related methods are provided. A mobile reverse PCR hood can include an air handler positioned in the hood top with the air handler configured to draw air out of an enclosed work area of the PCR hood when a sash of a front wall is in an open position. The air handler can include a prefilter, a high-efficiency particulate air (HEPA) filter, and a blower. The PCR hood can also include a UV light positioned within the enclosed work area and configured to sterilize the enclosed work area when activated for a set period of time.

### CROSS-REFERENCE TO RELATED APPLICATIONS

This application is a 371 Nationalization of National Application No.

PCT/US 2022/048593 filed Nov. 1, 2022 and claims the benefit of U.S. Provisional Patent Application No. 63/274,354, filed on Nov. 1, 2021, the entire contents of which are incorporated herein by reference.

### TECHNICAL FIELD

The present disclosure relates generally to mobile polymerase chain reaction (PCR) systems, hoods, and vehicles in which the PCR hoods can operate. More specifically, the invention relates to reverse PCR hoods that are useable within a mobile environment to allow for PCR tests to be conducted.

### BACKGROUND

Polymerase Chain Reaction, or PCR, is a method developed and used by biochemists when analyzing strands of DNA or RNA. In particular, PCR can be used to amplify, or multiply, a particular section of a strand of DNA or RNA to make studying of that segment easier. PCR has various applications ranging from medicine to environmental study to crime scene investigation.

Because cross-contamination during PCR work can produce inaccurate results, a clean environment is required. PCR workstations, also known as PCR hoods, that provide a clean environment in which to analyze and work with strands of DNA or RNA, have been developed to help make the process as error-free as possible. These PCR workstations or hoods prevent background and cross contamination by keeping all contaminants in the air away from samples.

PCR hoods are enclosed on three sides having a panel or sash on the front that allows the operator limited access to the workspace. To minimize contamination, either from the environment outside the PCR hood or between samples that have entered a respective workstation or hood, PCR hoods utilize high-efficiency particulate air (HEPA) filters to remove airborne contaminants found in a lab. Further, these PCR hoods traditionally operate as a laminar flow hood that provides uniform HEPA-filtered air flow across the work area by directing the clean air down over the work surface in unidirectional flow of air in parallel planes.

Traditionally, PCR hoods cannot be used in a mobile testing van due to contamination concerns. For example, concerns can relate to the typical airflow for a laminar flow hood, where air is pulled in through a filtering system and then flowing into the workspace in parallel planes down the wall and then exiting through the front access. Such an air flow system is more conducive to a stable clean environment such as in a cleanroom. Unfortunately, this has created a situation where PCR hoods are concentrated in certain areas, like universities or corporations or testing facilities. Recent global events such as the Coronavirus pandemic and the testing procedures and methods used to diagnosis related infections in humans has put a strain on traditional PCR practices and cause a recognition of underserved areas or other areas that may need additional access to safe and effective PCR analyzation and rapid testing not only in the United States but across the globe.

Therefore, a need exists for apparatuses, systems and methods that permit for mobile, effective, and sterile PCR analysis. Disclosed herein are one or more apparatuses, systems and methods that advantageously address these issues.

### SUMMARY

The foregoing and other aspects of the present subject matter will now be described in more detail with respect to other embodiments described herein. It should be appreciated that this subject matter can be embodied in different forms and should not be construed as limited to the embodiments set forth herein. Rather, these embodiments are provided so that this disclosure will be thorough and complete, and will fully convey the scope of the subject matter to those skilled in the art.

The present subject matter provides mobile PCR hoods, mobile PCR systems, and related methods. In particular, PCR hoods that operate in a reverse fashion, as compared to traditionally laminar flow hoods, in which air is pulled into the PCR hoods from the front access and up through one or more filtering apparatuses before blowing out of the PCR hood such that clean air flows back into the vehicle in which the PCR hood operates. The present subject matter also provides vehicles that create a clean environment in which reverse PCR hoods can operate. Methods related to the manufacture and use of the reverse PCR hoods, the mobile reverse hood systems, and the vehicles in which the reverse PCR hoods operate as disclosed herein are also provided.

In one embodiment, the disclosure provides a mobile PCR system for providing a mobile sterile work area for conducting PCR analysis. The system comprises a vehicle having an interior area that provides a clean environment; and a reverse PCR hood secured within the clean environment of the interior area of the vehicle. The reverse PCR hood comprises a base, two or more side walls secured to the base, the side walls extending upward from the base and forming a back and two sides of the PCR hood, a front wall that extends between the two sides of the PCR hood, the front wall comprising a sash that is movable between a closed position and an open position proximal to the base, a hood top secured to the side walls and front wall such that the base, the side walls, the front wall, and the hood top form an enclosed work area, an air handler positioned in the hood top, the air handler configured to draw air out of the enclosed work area when the sash of the front wall is in an open position with the air handler comprising a prefilter, a high-efficiency particulate air (HEPA) filter, and a blower, and an ultraviolet (UV) light bulb positioned within the enclosed work area and configured to provide UV light to sterilize the enclosed work area when activated for a set period of time.

In another embodiment the disclosure provides a reverse PCR hood comprising a base, two or more side walls secured to the base, the side walls extending upward from the base and forming a back and two sides of the PCR hood, a front wall that extends between the two side of the PCR hood, the front wall comprising a sash that is movable between a closed position and an open position proximal to the base, a hood top secured to the side walls and front wall such that the base, the side walls, the front wall, and the hood top form an enclosed work area, an air handler positioned in the hood top, the air handler configured to draw air out of the enclosed work area when the sash of the front wall is in an open position with the air handler comprising a prefilter, a high-efficiency particulate air (HEPA) filter, and a blower, and an UV light bulb positioned within the enclosed work area and configured to provide UV light to sterilize the enclosed work area when activated for a set period of time.

Other aspects of the disclosure will become apparent by consideration of the detailed description and accompanying drawings.

### BRIEF DESCRIPTION OF THE DRAWINGS

The previous summary and the following detailed descriptions are to be read in view of the drawings, which illustrate particular exemplary embodiments and features as briefly described below. The summary and detailed descriptions, however, are not limited to only those embodiments and features explicitly illustrated. A full and enabling disclosure of the present subject matter including the best mode thereof to one of ordinary skill in the art is set forth more particularly in the remainder of the specification, including reference to the accompanying figures, in which:

FIG. 1 illustrates a schematic side elevation view of an embodiment of a vehicle that houses embodiments of a PCR hood in a clean environment according to the present subject matter;

FIG. 2 illustrates a rear elevation view of an embodiment of a vehicle that houses embodiments of a PCR hood in a clean environment, the vehicle with its rear doors open to permit a view of the PCR hoods according to the present subject matter;

FIG. 3 illustrates an interior view of an embodiment of a vehicle that house embodiments of a PCR hood in a clean environment according to the present subject matter;

FIG. 4 illustrates a schematic partial cross-sectional side elevation view of the embodiment of the vehicle that houses the embodiments of the PCR hood in a clean environment according to FIG. 1;

FIG. 5 illustrates a schematic perspective view of an embodiment of a reverse PCR hood according to the present subject matter;

FIG. 6 illustrates a schematic partial cross-sectional side elevation view of an embodiment of a reverse PCR hood according to the present subject matter;

FIG. 7 illustrates an interior view of the embodiment of the vehicle that house the PCR hoods in a clean environment according to FIG. 1 with an operator conducting tests within one of PCR hoods according to the present subject matter;

FIG. 8 illustrates an image of an embodiment of a control panel used to control aspects an embodiment of a PCR hood according to the present subject matter; and

FIG. 9 illustrates a schematic partial cross-sectional side elevation view of an embodiment of an air lock exchange window for passing samples from the exterior of a vehicle that house embodiments of a PCR hood in a clean environment into the interior of the vehicle according to the present subject matter.

Repeat use of reference characters in the present specification and drawings is intended to represent the same or analogous features or elements of the present subject matter.

Other documentation and related information are provided herewith to further illustrate these, and other aspects of the subject matter disclosed herein and to further provide disclosure that is enabling to one of ordinary skill in the art.

### DETAILED DESCRIPTION

Before any embodiments of the invention are explained in detail, it is to be understood that the invention is not limited in its application to the details of construction and the arrangement of components set forth in the following description or illustrated in the following drawings. The invention is capable of other embodiments and of being practiced or of being carried out in various ways.

Reference now will be made to the embodiments of the present subject matter, one or more examples of which are set forth below. Each example is provided by way of an explanation of the present subject matter, not as a limitation. In fact, it will be apparent to those skilled in the art that various modifications and variations can be made in the present subject matter without departing from the scope or spirit of the present subject matter. For instance, features illustrated or described as one embodiment can be used on another embodiment to yield still a further embodiment. Thus, it is intended that the present subject matter cover such modifications and variations as come within the scope of the appended claims and their equivalents. It is to be understood by one of ordinary skill in the art that the present discussion is a description of exemplary embodiments only and is not intended as limiting the broader aspects of the present subject matter, which broader aspects are embodied in exemplary constructions.

Reference in this specification to “one embodiment” or “an embodiment” means that a particular feature, structure, or characteristic described in connection with the embodiment is included in at least one embodiment of the disclosure. The appearances of the phrase “in one embodiment” in various places in the specification are not necessarily all referring to the same embodiment, nor are separate or alternative embodiments mutually exclusive of other embodiments. Moreover, various features are described which may be exhibited by some embodiments and not by others. Similarly, various requirements are described which may be requirements for some embodiments but not for other embodiments.

The terms used in this specification generally have their ordinary meanings in the art, within the context of the disclosure, and in the specific context where each term is used. Certain terms that are used to describe the disclosure are discussed below, or elsewhere in the specification, to provide additional guidance to the practitioner regarding the description of the disclosure. For convenience, certain terms may be highlighted, for example using italics and/or quotation marks. The use of highlighting has no influence on the scope and meaning of a term; the scope and meaning of a term is the same, in the same context, whether or not it is highlighted. It will be appreciated that the same thing can be said in more than one way.

Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of other synonyms. The use of examples anywhere in this specification, including examples of any terms discussed herein, is illustrative only, and is not intended to further limit the scope and meaning of the disclosure or of any exemplified term. Likewise, the disclosure is not limited to various embodiments given in this specification.

Unless otherwise indicated, all numbers expressing quantities of components, conditions, and so forth used in the specification and claims are to be understood as being modified in all instances by the term “about.” Accordingly, unless indicated to the contrary, the numerical parameters set forth in the instant specification and attached claims are approximations that can vary depending upon the desired properties sought to be obtained by the presently disclosed subject matter.

Without intent to limit the scope of the disclosure, examples of instruments, apparatus, methods and their related results according to the embodiments of the present disclosure are given below. Note that titles or subtitles may be used in the examples for convenience of a reader, which in no way should limit the scope of the disclosure. Unless otherwise defined, all technical and scientific terms used herein have the same meaning as commonly understood by one of ordinary skill in the art to which this disclosure pertains. In the case of conflict, the present document, including definitions, will control.

Although the terms first, second, right, left, front, back, etc. may be used herein to describe various features, elements, components, regions, layers and/or sections, these features, elements, components, regions, layers and/or sections should not be limited by these terms.

These terms are only used to distinguish one feature, element, component, region, layer or section from another feature, element, component, region, layer or section. Thus, a first feature, element, component, region, layer or section discussed below could be termed a second feature, element, component, region, layer or section without departing from the teachings of the disclosure herein.

Similarly, when a layer or coating is being described in the present disclosure as “on” or “over” another layer or substrate, it is to be understood that the layers can either be directly contacting each other or have another layer or feature between the layers, unless expressly stated to the contrary. Thus, these terms are simply describing the relative position of the layers to each other and do not necessarily mean “on top of” since the relative position above or below depends upon the orientation of the device to the viewer.

Embodiments of the subject matter of the disclosure are described herein with reference to schematic illustrations of embodiments that may be idealized. As such, variations from the shapes and/or positions of features, elements or components within the illustrations as a result of, for example but not limited to, user preferences, manufacturing techniques and/or tolerances are expected. Shapes, sizes and/or positions of features, elements or components illustrated in the figures may also be magnified, minimized, exaggerated, shifted or simplified to facilitate explanation of the subject matter disclosed herein. Thus, the features, elements or components illustrated in the figures are schematic in nature and their shapes and/or positions are not intended to illustrate the precise configuration of the subject matter and are not intended to limit the scope of the subject matter disclosed herein.

As used herein, the term “clean environment” means an enclosed area that's controlled environmentally to control atmospheric contamination, temperature, pressure and, often, humidity to provide a safe and non-contaminated workspace for testing, analyzing, amplifying, or multiplying biological and or chemical substances.

As disclosed herein, mobile PCR systems, mobile reverse PCR hoods and vehicles that provide a clean environment to conduct PCR testing as well as methods for using and making such mobile PCR systems, mobile reverse PCR hood, and clean environment vehicles are provided. The present subject matter provides a mobile PCR system for providing a mobile sterile work area for conducting PCR testing. The mobile PCR system can comprise a vehicle having an interior area that provides a clean environment. The mobile system can also comprise at least one reverse PCR hood secured within the clean environment of the interior area of the vehicle. The reverse PCR hood can comprise a base and two or more side walls secured to the base. The side walls extend upward from the base and forming a back and two sides of the PCR hood. The PCR hood can also comprise a front wall that extends between the two side of the PCR hood. The front wall can comprise a sash that is movable between a closed position and an open position proximal to the base. Additionally, the PCR hood can comprise a hood top secured to the side walls and front wall such that the base, the side walls, the front wall, and the hood top form an enclosed work area. The PCR hood can also comprise an air handler positioned in the hood top. The air handler can be configured to draw air out of the enclosed work area when the sash of the front wall is in an open position. The air handler can comprise a prefilter, a high-efficiency particulate air (HEPA) filter, and a blower. Further, the PCR hood can comprise an ultraviolet (UV) light positioned within the enclosed work area and configured to sterilize the enclosed work area within the PCR hood when activated for a set period of time. Embodiments of the mobile PCR systems, mobile reverse PCR hoods and vehicles that provide a clean environment are described in further detail below.

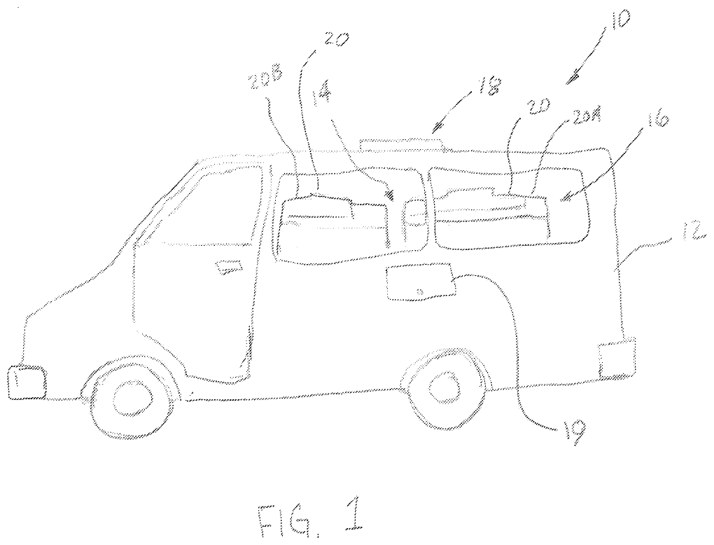

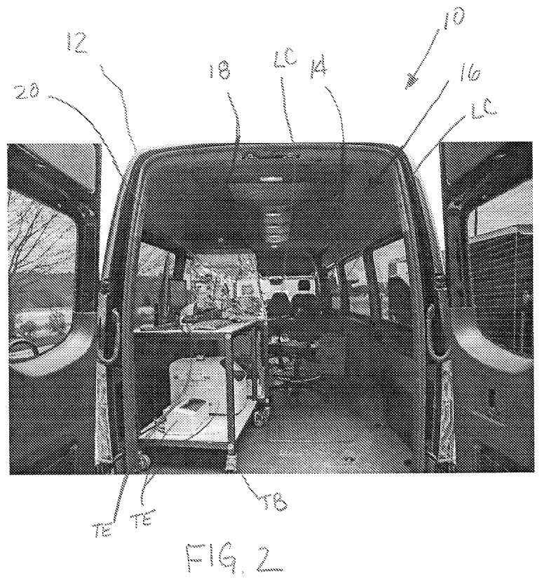

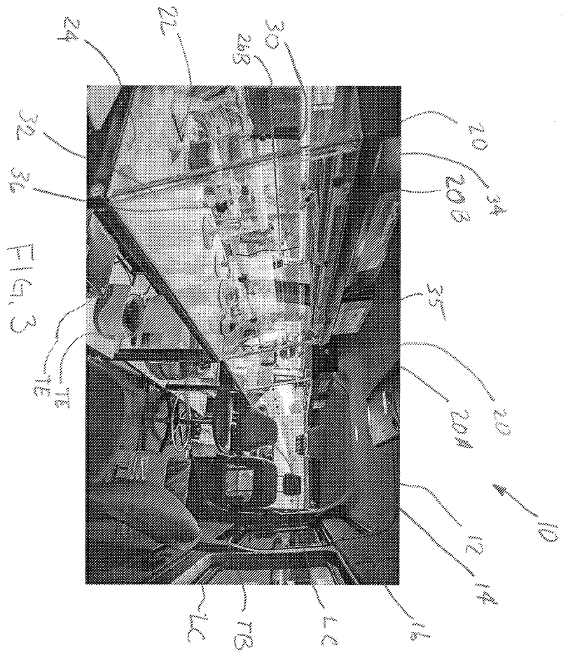

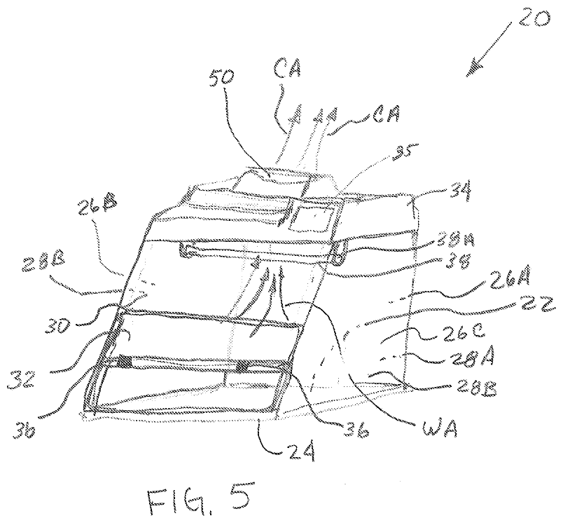

Referring now to FIGS. 1-9, example embodiments of mobile PCR systems that can include embodiments of a vehicle providing a clean environment and embodiments of a mobile reverse PCR hood are provided. FIG. 1 illustrates an embodiment of a mobile PCR system 10. As shown, the mobile PCR system 10 comprises a customized vehicle 12 that can have a clean environment 14 within an interior 16 of the vehicle 12. In some embodiments, the vehicle 12 can be a van or a trailer that permits operators to work inside the clean environment 14 when PCR analysis is to be performed as shown in FIGS. 2 and 3. The mobile PCR system 10 can also comprise one or more PCR hoods 20, such as PCR hoods 20A, 20B, that are workstations securely positioned in the interior 16 in the clean environment 14 of the vehicle 12 and provide an enclosed workspace, or work area, 22 in which to perform the PCR analysis. The PCR hoods 20A, 20B, as will be described in greater detail below, can be reverse PCR hoods in which air is drawn out of the enclosed work area 22 and cleaned before being released as clean air back in the clean environment 14 of the vehicle 12 during use. The vehicle 12 can also comprise a vehicle air handler 18. The vehicle air handler 18 is provided to ensure a cleaner and particle-free environment inside the interior 16 of the vehicle 12 to create the clean environment. The air handler 18 can comprise one or more HEPA filters that are effective enough to capture all the micro-size particles in the air that enter the cleanroom to assure a cleaner and particle-free environment inside the clean environment. In some embodiments, the air handler 18 can comprise a prefilter, a blower, and a HEPA filter that is used to clean the air so that the air exiting the vehicle air handler is clean air that flows into the clean environment of the vehicle.

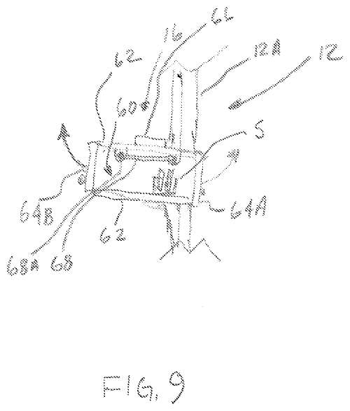

Additionally, in some embodiments, the vehicle 12 can also comprise an exchange window 19 for delivery of samples to be tested into the clean environment to help minimize exposure of the clean environment to possible contaminants from the outside world. In some embodiments, the exchange window 19 can comprise an airlock exchange window for passing samples from outside the vehicle 12 into the clean environment 14. For example, in some embodiments, as shown in FIG. 9, an airlock exchange window 19 can be in a side wall 12A of a vehicle 12. The exchange window 19 can comprise a chamber 60 formed by walls 62 with an entry door 64A on the exterior of the vehicle and an interior door 64B on the interior 16 of the vehicle 12. The air lock exchange window can have an air handler 66 that can be used to provide clean air such as a clean air shower or, alternatively, can operate as a vacuum to remove contaminates. Additionally, a light fixture 68A can be provided in the chamber 60 with an ultraviolet (UV) light bulb 68 therein for providing UV light to sterilize the chamber 60. In some embodiments, the samples can be passed into the interior of the vehicle 12 using other methods, including passing the samples through a cracked window or door.

As stated above, the interior 16 of the vehicle 12 can be sized to provide room for equipment such as the reverse PCR hood 20 and other testing equipment TE which can reside on testing table TB and still permit the lab operators to comfortably move within the interior 16 to perform the tasks that are required to conduct the PCR testing and analysis. For example, the interior 16 can be suitably sized for two operators to sit in lab chairs LC and work at the workstations, or PCR hoods 20A and 20B and provide room for a third operator to move freely in the interior 16 of the vehicle 12 to collect samples and/or operate other testing equipment TE as needed.

To operate as a clean environment 14, the vehicle 12 provides the vehicle air handler 18 that removes contamination from the air by removing up to at least about 99% of the particulate matter in the air that enters the interior 16 of the vehicle 12 up to about 0.3 microns in size. In some embodiments, the air handler 18 can comprise one or more HVAC units that filters the air through one or more HEPA filters and can provide air into the interior 16 of the vehicle in a laminar flow. Such HVAC units that use the HEPA filters can provide clean air that is about 99.99% free or greater of particles of about 0.3 microns or larger in size. The interior of the vehicle 12 can also include ultraviolet (UV) lighting that can be used to sterilize the interior 16 before entry of the operators. The operators can performance procedures to decontaminate themselves before entering the vehicle 12 including using cleanroom garments and use sticky mats. Cleanroom garments are garments made of synthetic fabrics such as Nylon, Dacron, Tyvek and others. They are lint free and limit contamination from personnel. Sticky mats are multiple layers of treated film with adhesive to clean shoe bottoms and sides before entering the room and can be located at an entry point for operators entering the vehicle 12.

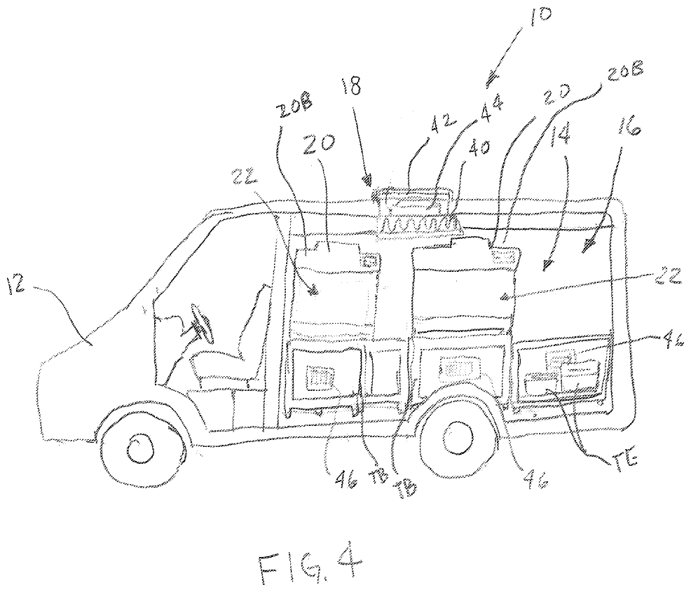

In some embodiments as shown in FIG. 4, the vehicle air handler 18 can comprise a HEPA filter 40 as well as prefilter 42 that can be used to prefilter the air before passing through a fan, or blower, 44. Depending on the configuration of the air handler and its intended operation, the air can be provided and moved differently. In some embodiments, the air handler system can operate in reverse. As shown in FIG. 4, the air handler system can provide clean air into the interior of the vehicle by filtering outside air. The air handler 18 can draw air from outside of the vehicle 12, clean it and pump the clean air into interior 16 of the vehicle 12 to create the clean environment 14. The blower 44 can draw air through the prefilter 42 which can comprise one or more electrostatic particulate filters which can have about a 95% efficiency or greater for removing particulate matter down to about 0.3 microns in diameter. The blower 44 can then push the prefiltered air through the HEPA filter 40 to further sterilize the air by trapping any particulate matter of about 0.3 microns in diameter therein as the air passes through the HEPA filter 40. For example, the air handler 18 can create a laminar flow of clean air within the interior 16 of the vehicle 12. In some embodiments, the air handler 18 can comprise an exhaust system 46 to provide a continuous flow of clean air. For example, in some embodiments, the exhaust system 46 can be located at a base of the vehicle 12 to facilitate the creation of a laminar flow of clean air to create the clean environment 14 in which the reverse PCR hoods can operate.

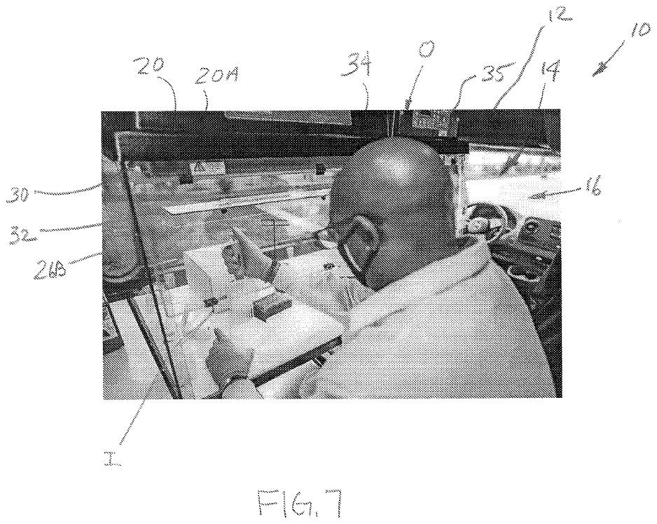

As shown in more detail in FIGS. 3-6, a reverse PCR hood 20 can be provided that can comprise a base 24 and two or more side walls 26A, 26B, 26C secured to the base 24. The side walls 26A, 26B, 26C can extend upward from the base 24 and forming a back 28A and two sides 28B of the PCR hood 20. The PCR hood 20 can also comprise a front wall 30 that extends between the two side 28B of the PCR hood 20. The front wall 30 can comprise a sash 32 that is movable between a closed position and an open position proximal to the base 24. Additionally, the PCR hood 20 can comprise a hood top 34 secured to the side walls and front wall 30 such that the base 24, the side walls 26A, 26B, 26C, the front wall 30, and the hood top 34 form an enclosed work area 22. In some embodiments as shown, the side walls 26A, 26B, 26C can comprise a back wall 26A, a first side wall 26B and a second side wall 26C and the front wall 30 can be secured to and can extend between the first side wall 26B and the second side wall 26C. The front edges of the first side wall 26B and second side wall 26C can be slanted so that the front wall 30 resides at an angle for better viewing therethrough by an operator O (See FIG. 7). The sash 32 of the front wall 30 can be attached to the stationary portion 30A of the front wall 30 by hinges 36 so that the sash 32 is movable between the open position that forms an access opening in the front wall 30 where an operator can access the enclosed work area 22 and a closed position where the front wall 30 is securely closed preventing access to the enclosed work area 22.

The side walls 26A, 26B, 26C and the front wall 30 and sash 32 can comprise one or more polymer materials that is strong and sturdy but is transparent or translucent to allow the enclosed work area 22 to be viewable from outside the PCR hood 20. For example, side walls 26A, 26B, 26C and the front wall 30 and sash 32 can comprise a polycarbonate, such as LEXAN sold by SBIC. Similarly, the base 24 and the hood top 34 can also comprise one or more polymer materials. For example, in some embodiments, the hood top 34 can comprise an acrylonitrile butadiene styrene (ABS) and the base 24 can comprise a structural polypropylene.

The side walls 26A, 26B, 26C and the front wall 30 can be tightly connected and built in such a way, without gaps, pores, or joints, to avoid contamination, either from the external environment into the enclosed work area 22 of the PCR hood 20, or from the enclosed work area 22 of the PCR hood 20 out to the external environment.

Further, the PCR hood 20 can comprise a light fixture 38A and an ultraviolet (UV) light bulb 38 positioned within the enclosed work area 22. Through the light fixture 38A, the UV light bulb 38 can shine UV light within the enclosed work area 22 to sterilize the enclosed work area 22 within the PCR hood 20 when activated for a set period of time. The set period of time of exposure of the enclosed work area 22 to the UV light can vary. For example, in some embodiments, the set period of time can be about 15 minutes. To prevent under wanted or undesirable exposure to UV light, when the sash 32 is moved to the open position, the UV light bulb 38 can be automatically turned off.

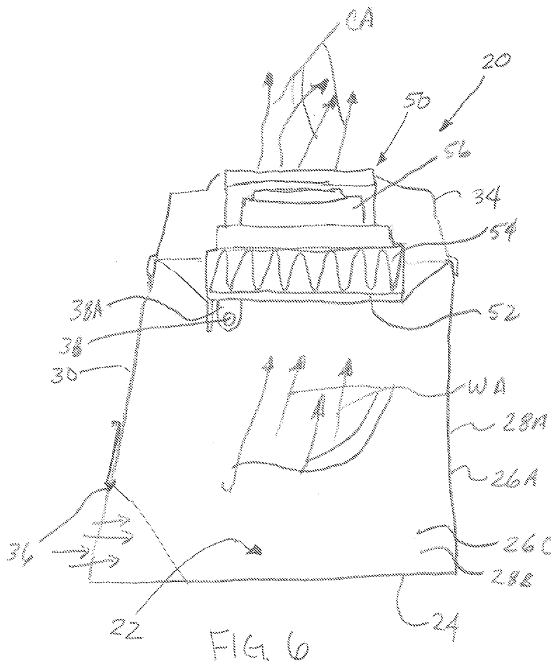

The PCR hood 20 can also comprise an air handler 50 positioned in the hood top 34. The air handler 50 can be configured to draw air WA out of the enclosed work area 22 when the sash 32 of the front wall 30 is in an open position. The air handler 50 can comprise a prefilter 52, a high-efficiency particulate air (HEPA) filter 54, and a blower 56. As shown in FIG. 6, the air handler 50 can be configured with the prefilter 52 positioned proximate to the enclosed work area 22 with the HEPA filter 54 positioned between the prefilter 52 and the blower 56 such that the blower 56 draw air WA first through the prefilter 52 and then the HEPA filter 54 before the clean air CA passes through the blower 56 to be pushed out into the clean environment 14 in the interior 16 of the vehicle 12. For example, when the sash 32 of the front wall 30 is in an open position, the blower 56 can be turned on automatically drawing the air WA through the prefilter 52 and into the HEPA filter 54 before the now clean air CA passes through and exits the blower 56 such that the clean air CA flows into the clean environment 14 of the vehicle 12.

Once the sash 32 of the front wall 30 is moved to the closed position, the blower 56 can be turned off automatically. In some embodiments, the prefilter 52 comprises a filtrate electrostatic particulate filter having about a 97% efficiency for particulates down to about 0.3 microns in diameter. In some embodiments, the air passing through the air handler 50 exits the blower 56 as clean air CA with about 99% or greater of particles 0.3 microns or larger in size removed. In some embodiments, the air passing through the air handler 50 exits the blower 56 as clean air CA with about 99.99% or greater of particles of about 0.3 microns or larger in size removed.

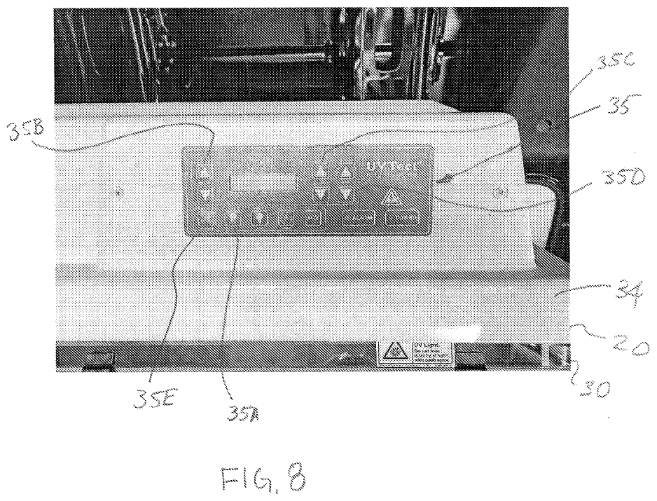

In some embodiments as shown, a controller 35 as shown in FIG. 8 can be secured on the hood top 30. The controller 35 can be configured to control the operation of the air handler 50 and the light fixture 38A in which the UV light bulb 38 resides. For example, in some embodiments, the light fixture 38A and UV light bulb 38 can be activated by the controller 35 such that the UV light remains on to sterilize the enclosed work area 22 for a set period of time before turning off. Similarly, the controller 35 can be used to control when the blower 56 of the air handler 50 is turned on and off and the speed at which the blower 56 operates. The volume of filtered air can be about 200 CFM.

The PCR hood 20 is designed to provide sterilization of instrumentation I and the enclosed work area 22 before the process and to protect the operator O as shown in FIG. 7 from particulate matter during the processing of samples. The light fixture 38A, the UV light bulb 38 and the UV light that is created is an integral part of the process but must be handled with care. To begin the sterilization period (not shown), the sash 32 can be closed all the way. The UV on-switch 35A can be pressed on the controller 35. The light fixture 38A will power the UV light bulb 38 and the UV light will be turned on and can automatically turn off after a set period of time, such as about 15 minutes. The controller can be programmed so that, when the sash 32 is raised, the blower 56 will turn on automatically and a fluorescent light turns on automatically, while the UV light bulb 38 is turned off.

As shown in FIG. 8, the Controller 35 can give the operator O a variety of options with the ease of fingertip control. The controller 35 can have mode keys 358 that allows the operator O to view different programming menus and select keys 35C that allows the operator O to view different various other menu options within a certain menu. The set keys 35D can allow the operator O to toggle or change specific options with a certain menu. Additionally, quick keys 35E can allow the operator O to turn on and off the different features such as the blower 56, the light fixture 38A, etc. The different programming menus can include a main menu, a blower control menu, a timer, a UV light control menu, a low air flow alarm and gas detector settings menu, a HEPA filter alarm menu, and filter menu for example. Scheduled maintenance and replacement are important for both the prefilters 52 and the HEPA filters 54.

These and other modifications and variations to the present subject matter may be practiced by those of ordinary skill in the art, without departing from the spirit and scope of the present disclosure, which is more particularly set forth herein above. In addition, it should be understood the aspects of the various embodiments may be interchanged both in whole and in part. Furthermore, those of ordinary skill in the art will appreciate that the foregoing description is by way of example only and is not intended to limit the present disclosure.

1. A mobile PCR system for providing a mobile sterile work area for conducting PCR analysis, the system comprising:
a vehicle having an interior area that provides a clean environment; and
a reverse PCR hood secured within the clean environment of the interior area of the vehicle, the reverse PCR hood comprising:
a base;
two or more side walls secured to the base, the side walls extending upward from the base and forming a back and two sides of the PCR hood;
a front wall that extends between the two sides of the PCR hood, the front wall comprising a sash that is movable between a closed position and an open position proximal to the base;
a hood top secured to the side walls and front wall such that the base, the side walls, the front wall, and the hood top form an enclosed work area;
an air handler positioned in the hood top, the air handler configured to draw air out of the enclosed work area when the sash of the front wall is in an open position with the air handler comprising a prefilter, a high-efficiency particulate air (HEPA) filter, and a blower; and
an ultraviolet (UV) light bulb positioned within the enclosed work area and configured to provide UV light to sterilize the enclosed work area when activated for a set period of time.

2. The mobile PCR system according to claim 1, wherein the two or more side walls of the PCR hood comprise a back wall, a first side wall and a second side wall and the front wall is secured to and extends between the first side wall and the second side wall.

3. The mobile PCR system according to claim 2, wherein the sash movably hinged to a stationary portion of the front wall so that the sash is movable between the open position that forms an access opening in the front wall where an operator can access the enclosed work area and a closed position where the front wall is securely closed preventing access to the enclosed work area.

4. The mobile PCR system according to claim 1, wherein the air handler is configured with the prefilter positioned proximate to the enclosed work area with the HEPA filter positioned between the prefilter and the blower such that when, the sash of the front wall is in an open position, the blower is turned on drawing air through the prefilter and into the HEPA filter before the air passes through and exits the blower.

5. The mobile PCR system according to claim 4, wherein the air exiting the blower is clean air that flows into the clean environment of the vehicle.

6. The mobile PCR system according to claim 5, wherein the air passing through the air handler exits the blower as clean air with about 99% or greater of particles 0.3 microns or larger in size removed.

7. The mobile PCR system according to claim 5, wherein the air passing through the air handler exits the blower as clean air with about 99.99% or greater of particles of about 0.3 microns or larger in size removed.

8. The mobile PCR system according to claim 5, wherein the prefilter comprises a filtrate electrostatic particulate filter having about a 97% efficiency for particulates down to about 0.3 microns in diameter.

9. The mobile PCR system according to claim 1, further comprising a controller configured to control the operation of the air handler and a light fixture in which the UV light bulb resides.

10. The mobile PCR system according to claim 9, wherein the UV light is activatable by the controller such that the UV remains on to sterilize the enclosed work area for a set period of time before turning off.

11. The mobile PCR system according to claim 10, wherein the set period of time is about 15 minutes.

12. The mobile PCR system according to claim 9, wherein, when the sash is moved to the open position, the UV light will turn off.

13. The mobile PCR system according to claim 1, wherein the vehicle comprises a vehicle air handler that comprises a prefilter, a blower, and a HEPA filter.

14. The mobile PCR system according to claim 13, wherein the air exiting the vehicle air handler is clean air that flows into the clean environment of the vehicle.

15. The mobile PCR system according to claim 1, wherein the vehicle comprises an airlock exchange window for passing samples from outside the vehicle into the clean environment.

16-27. (Canceled)
